(19) United States
(12) Patent Application Publication
(10) Pub. No.: US 2021/0022831 A1
(43) Pub. Date: Jan. 28, 2021

(54) ENDODONTICS SURGICAL STENT

(71) Applicant: CORNERSTONE SPECIALTY PRODUCTS, LLC, Irvine, CA (US)

(72) Inventors: Hamid Reza ABEDI, Irvine, CA (US); Mohammadreza FOROUGHI, Irvine, CA (US); Muazz ZENDAKI, Mission Viejo, CA (US)

(21) Appl. No.: 17/037,374

(22) Filed: Sep. 29, 2020

Related U.S. Application Data
(63) Continuation-in-part of application No. 16/365,468, filed on Mar. 26, 2019.

(51) Int. Cl. A61C 5/82; A61C 5/42; A61B 17/3209; A61C 5/50; A61C 1/08

(52) U.S. Cl. CPC A61C 5/82; A61C 5/42; A61B 2017/320052; A61C 5/50; A61C 1/082; A61B 17/3209

(57) ABSTRACT

A hard tissue stent, including an access hole formed in one side, may be placed over one or more of the patient's teeth where the access hole is accurately aligned at a location for drilling to, for example, remove infection. A soft tissue stent may be placed over one or more of the patient's teeth, where the stent can include a mark to cut to create an access flap in the soft tissue. By keeping the flap attached, the surgeon can more easily return the flap to the proper position. The attached surgical guide permits access any area of the jaw and tooth and implanted device. With insertion of a sleeve with custom bores into the guide, the dental surgeon can work in a dry field and accurately manipulate the bony crypt and tooth for many different purposes including root-end preparation, filling and repair of local perforations and cracks.

### CROSS-REFERENCE TO RELATED APPLICATIONS

This application is a continuation-in-part of U.S. patent application Ser. No. 16/365,468, filed Mar. 26, 2019, the contents of which are herein incorporated by reference.

### BACKGROUND OF THE INVENTION

### 1. Field of the Invention

Embodiments of the invention relates generally to dental instruments. More particularly, the invention relates to dental surgical stents that fit a patient's tooth or teeth to help guide a dentist during a procedure such as a root canal, apicoectomy, or the like.

### 2. Description of Prior Art and Related Information

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

The primary aim of any endodontic treatment is to disinfect the root canal system in order to reduce the bacterial load as much as possible and to seal the system to prevent ingress or egress of bacteria or their byproducts. A root canal procedure often accesses the root from the top of the tooth to remove infection therein. In some applications, a dental professional, such as an endodontist, would need to access infection, cracks or other anomalies of the root sub gingivally. A flap may be made of the soft tissue to provide access to the bone and a tool may be used to remove the bone to gain access to the area of interest. The infection may be removed, and the flap replaced to heal. In some circumstances, the root tip may be removed, a root-end cavity prepared and a biocompatible root end filling placed.

Often, once a flap is cut, it is difficult to handle and maintain it in its proper position during the surgical procedure. The surgeon needs to keep the flap out of the way during the surgery and properly position the flap back in place thereafter. The soft tissue flap may be difficult to handle after it is cut. There is bleeding and fluid also in the surgical crypt making visualization difficult in some cases.

Moreover, to access the infection, the surgeon may need to remove a substantial amount of bone to not only gain access but also to visually ensure the position and depth of hard tissue is correct.

In view of the foregoing, there is a need for dental surgical stents, that may secure the flap during surgery as well as provide a hard tissue drilling location during dental surgery, and a surgical sleeve to ensure accurate access to any area of the root and crypt to allow manipulation whilst working in a dry field.

### SUMMARY OF THE INVENTION

In some embodiments, the present invention provides a dental surgical system comprising a stent body configured to drape over at least one tooth; a surgical guide attached to one side of the stent body, the surgical guide positioned over at least a portion of a gum region of a tooth; an opening disposed through the surgical guide; a sleeve configured to fit into the opening of the surgical guide; and a bore through the sleeve, the bore communicating a proximal end of the sleeve with a side of the sleeve adjacent to a distal end thereof.

Embodiments of the present invention further provide method for accessing a root canal of a tooth comprising draping a stent body at least one tooth, the stent body having a surgical guide attached to one side thereof, the surgical guide positioned over at least a portion of a gum region of the tooth; creating an incision through tissue near a desired access site; flapping the tissue at the incision; positioning the surgical guide under the flap; positioning a trephine bur through an opening in the surgical guide; forming an access opening through tissue and bone with the trephine bur; terminating the access opening at any point in the crypt or root which may be the base of a root of the tooth; and inserting a sleeve into the access opening, the sleeve having a bore through the sleeve, the bore communicating a proximal end of the sleeve with a side of the sleeve adjacent a distal end thereof, the bore positioned adjacent the root canal of the tooth.

Embodiments of the present invention also provide a method for treating a tooth, comprising draping a stent body at least one tooth, the stent body having a surgical guide attached to one side thereof, the surgical guide positioned over at least a portion of a gum region of the tooth; creating a first incision and a second incision at each side of the surgical guide; removing the surgical guide to provide a further incision connecting the first incision with the second incision; flapping the tissue at the incision with a tissue separator, attached to the surgical guide opposite the stent body, as the surgical guide is positioned under the flap; positioning a trephine bur through an opening in the surgical guide; forming an access opening through tissue and bone with the trephine bur; terminating the access opening at any location on the root; inserting a sleeve into the access opening, the sleeve having a bore through the sleeve, the bore communicating a proximal end of the sleeve with a side of the sleeve adjacent a distal end thereof, the bore positioned adjacent to the root of the tooth; inserting a dental tool, such as a file, bur, sonic, ultrasonic, piezo, laser root preparation tips, or the like, through the bore to manipulate the root/root canal; and inserting a retro filling material through the bore to be applied into the root canal.

These and other features, aspects and advantages of the present invention will become better understood with reference to the following drawings, description and claims.

### BRIEF DESCRIPTION OF THE DRAWINGS

Some embodiments of the present invention are illustrated as an example and are not limited by the figures of the accompanying drawings, in which like references may indicate similar elements.

FIG. 1 illustrates a perspective view of a sleeve for placement in a surgical guide;

FIG. 2A illustrates a left side view of a stent part similar to a nut, including two sharp parts that, after screwing in the sleeve, lifts and keeps the gingiva;

FIG. 2B illustrates a perspective view of a stent part similar to a nut, including two sharp parts that, after screwing in the sleeve, lifts and keeps the gingiva;

FIG. 3 illustrates a screw that can be attached to the sleeve when using a round bur or probe for dental purposes;

FIG. 4A illustrates a top view of the assembled stent for round bur and probe application;

FIG. 4B illustrates a bottom view of the assembled stent for round bur and probe application;

FIG. 5 illustrates the assembled stent for a trephine bur application;

FIG. 6 illustrates the assembled stent for a round bur application, flap lines and a finishing step;

FIG. 7 illustrates a schematic view of a stent attached on teeth;

FIG. 8 illustrates a schematic view of a stent attached on teeth;

FIG. 9 illustrates a schematic view of a stent attached on teeth having an access opening formed therein;

FIG. 10 illustrates a front view of a stent attached on teeth having a surgical guide and tissue separator attached thereto;

FIG. 11 illustrates a front view of the stent of FIG. 10 with the tissue separator moved downward to separate tissue along an incision line;

FIG. 12 illustrates a dental tool having a stopper integrated therewith for providing root access through the surgical guide of FIG. 10;

FIG. 13 illustrates a side view of the stent in position and the dental trephine drill inserted through the surgical guide to providing access to a tooth root, according to an exemplary embodiment of the present invention;

FIG. 14 illustrates the male alignment member separated from the surgical guide of FIG. 13;

FIG. 15 illustrates a male alignment member inserted into the surgical guide of FIG. 10;

FIG. 16 illustrates a side view of the male alignment member being inserted into the surgical guide;

FIG. 17 illustrates proper positioning of the male alignment member at the base of a tooth root;

FIG. 18 illustrates proper positioning of the male alignment member at the base of a tooth root, with filling material inserted into the tooth's root canal;

FIG. 19 illustrates proper position of a male alignment member for repair at a side location of a root, such as for repair of a cracked root, for example; and

FIG. 20 illustrates a flow chart describing a method according to an exemplary embodiment of the present invention.

Unless otherwise indicated illustrations in the figures are not necessarily drawn to scale.

The invention and its various embodiments can now be better understood by turning to the following detailed description wherein illustrated embodiments are described. It is to be expressly understood that the illustrated embodiments are set forth as examples and not by way of limitations on the invention as ultimately defined in the claims.

### DETAILED DESCRIPTION OF THE PREFERRED EMBODIMENTS AND BEST MODE OF INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

As is well known to those skilled in the art, many careful considerations and compromises typically must be made when designing for the optimal configuration of a commercial implementation of any device, and in particular, the embodiments of the present invention. A commercial implementation in accordance with the spirit and teachings of the present invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Broadly, embodiments of the present invention provide a dental surgical stent that can be used to assist a dental surgeon. A hard tissue stent can include an access hole formed in one side and may be placed over one or more of the patient's teeth where the access hole is accurately aligned at a location for drilling to, for example, to repair a crack, perforation, resorptive defect, removing infected root end, foreign material or remove infection. A soft tissue stent may be placed over one or more of the patient's teeth, where the stent can include a mark positioned where the surgeon is to cut to create an access flap in the soft tissue. By keeping the flap attached to the semi-circular window of the soft tissue stent, the surgeon can more easily return the flap to the proper position and protect it from damage during the dental procedure.

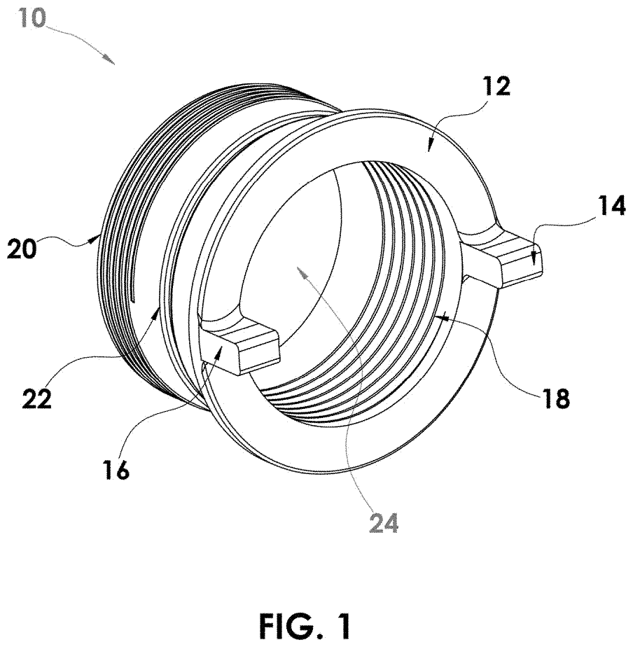

Referring now to FIGS. 1 through 4B, a sleeve 10 can include a front face 12 having one or more handles 14, 16 extending therefrom. The handles 14, 16 may extend away from the front face 12, as shown, to aid a user in rotating the sleeve 10 and the nut 30 to cause opening of the flap on the soft tissue of a patient, as discussed below.

The sleeve 10 can include inside threads 18 (also referred to as female threads 18) that begin at the front face 12 and extend downward toward a back face thereof. The sleeve 10 can include outside threads 20 (also referred to as male threads 20) extending from the back face of the sleeve 10 as shown in FIG. 1.

A groove 22 may be disposed about an exterior circumference of the sleeve 10 at a longitudinally central portion thereof. The groove 22 may be helpful to minimize the concentration of compressive stress during use of the surgical guide as discussed in greater detail below.

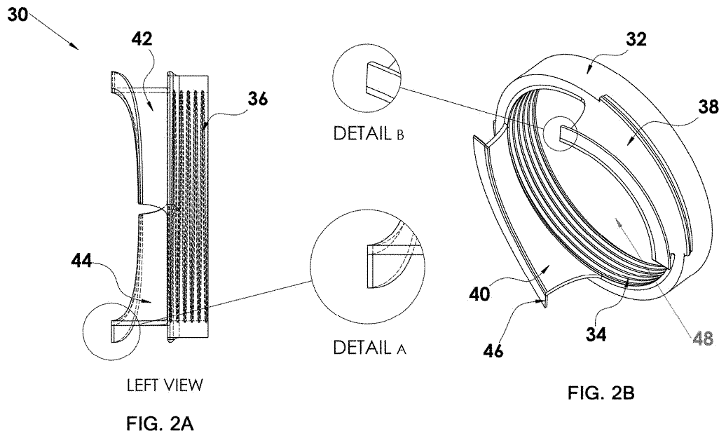

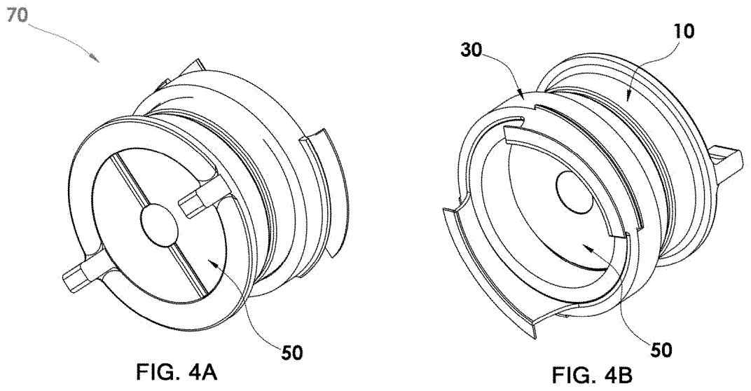

A nut 30, as shown in FIGS. 2A and 2B, can include a cylindrical exterior 32 having female threads 34 positioned on an interior of the cylindrical exterior 32. The female threads 34 can mate with the male threads 20 of the sleeve 10 when the front end 36 of the nut 30 is threaded onto the sleeve 10, thereby attaching the nut 30 to the sleeve 10 as shown in FIGS. 4A and 4B. A first side extension, having an inner surface 40 and an outer surface 44 may extend from the cylindrical exterior 32, typically along from about 35 to 55 degrees of the back end of the cylindrical exterior 32. A second side extension, having an inner surface 42 and an outer surface 38, may extend from the cylindrical exterior 32, typically along from about 35 to 55 degrees of the back end of the cylindrical exterior 32. The first and second side extensions may be disposed equally spaced apart from each other. Distal ends (distal relative to the cylindrical exterior 32) may be bent outward (away from a central opening 48 of the nut 30) at an angle, typically about 90 degrees. Sharp parts 46 may be disposed at distal ends of the first and second side extensions at the bent portions. In some embodiments, the first and second side extensions may be longer (extend farther away from the cylindrical exterior 32) adjacent the sharp parts 46, providing an edge to cut through soft tissue when the nut 32 is placed against the soft tissue and rotated.

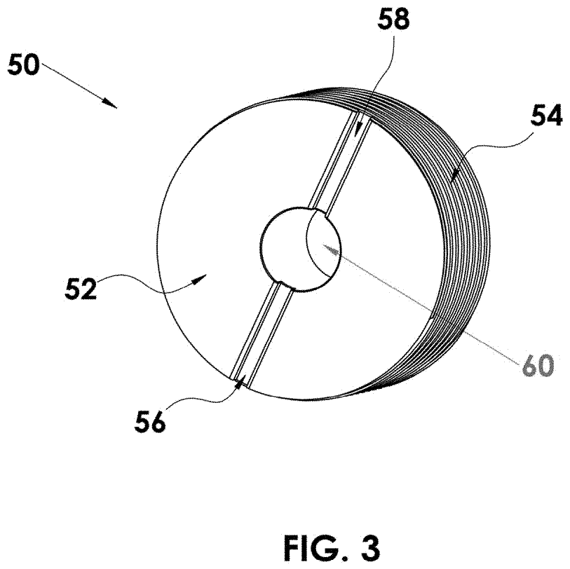

A screw 50, as shown in FIG. 3, can include an exterior thread 54 (also referred to as mail thread 54 or screw male thread 54) that extends along an entirety of the external circumference of the screw 50. The screw 50 can include a central opening 60 for use of the surgical guide, as discussed below. The screw 50 may include a front face 52 having channels 56, 58 formed therein. The channels 56, 58 can extend from the central hole 60 to the exterior of the screw 50. The channels 56, 58 may be useful for turning the screw 50 for positioning inside the sleeve 10, as discussed below. As discussed below, the screw 50 may be useful for making the opening 24 of the sleeve 10 smaller, for certain procedures, in essence reducing the opening from the size of the opening 24 to the size of the opening 60.

FIGS. 4A and 4B illustrate an assembly 70 of the screw 50, nut 30 and sleeve 10.

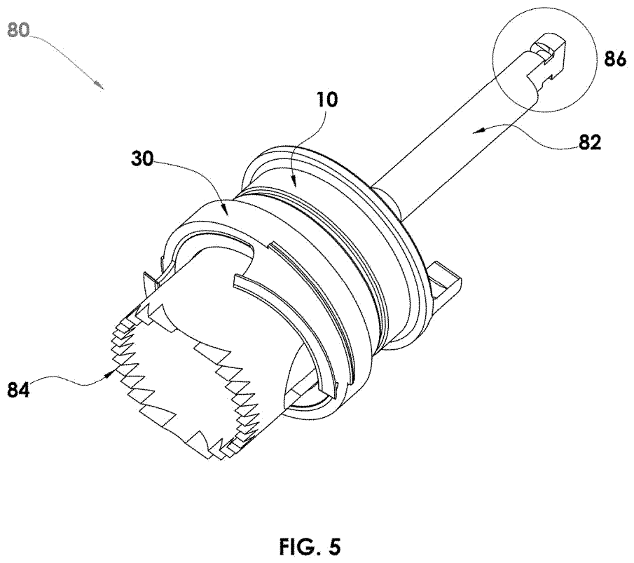

Referring now to FIG. 5, a dental assembly 80 can include the sleeve 10 assembled with the nut 30. The nut 30 may be assembled with the sleeve 10 and the sleeve 10 may be rotated, which, in turn, rotates the nut 30. The sharp parts 46 may press against soft tissue (such as the gums, or gingiva) and, as rotated, form a flap of tissue that may be lifted and kept. In the embodiment of FIG. 5, a trephine bur 84 may be attached to a shaft 82 having a dental tool attachment end 86 that provides a motive force to turn the trephine bur 84. As seen in this example, the sleeve 10 may not include the screw 50 so that the opening 24 of the sleeve 10 can receive the trephine bur 84 therethrough.

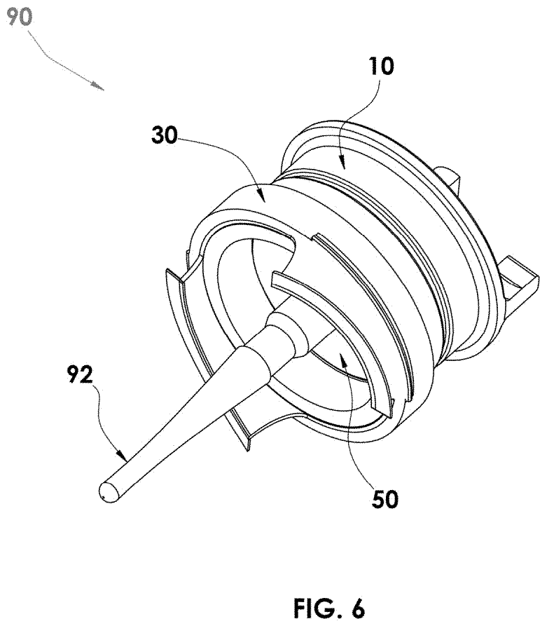

Referring now to FIG. 6, a dental assembly 80 can include the sleeve 10 assembled with the nut 30, with the screw 50 inserted in the sleeve 10. A round bur 92 may be inserted through the opening 60 of the nut 50, as shown, to provide round bur application and finishing steps.

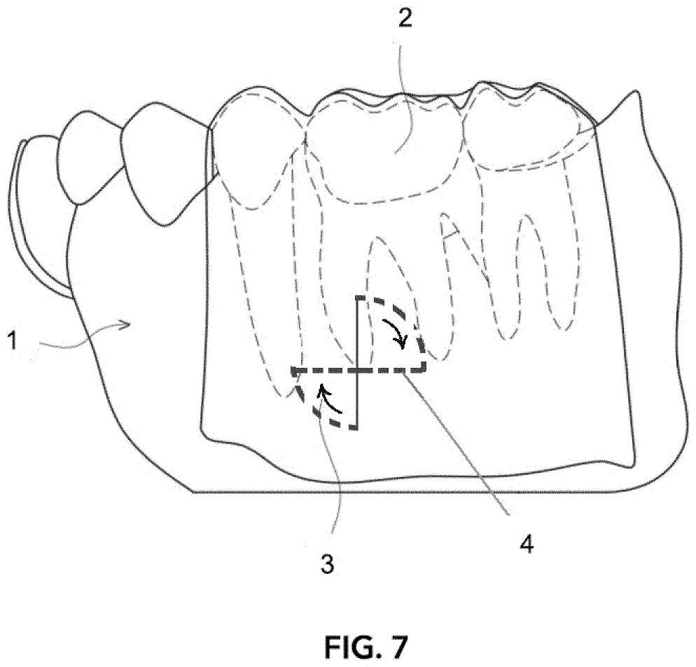

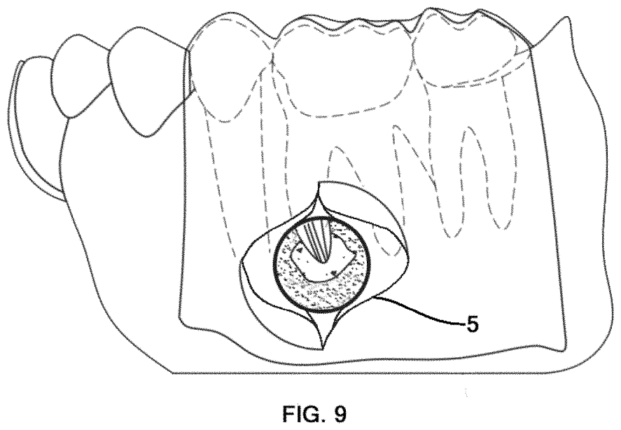

Referring to FIG. 7, a stent can be draped over at least one tooth 2, where side members of the stent body extends downward to cover at least a portion of the gum region of at least one tooth. The stent body can include an access opening 5, as shown in FIG. 9, formed in the side member to provide access positioned in a location for drilling hard tissue there between. In some embodiments, the stent body can include an incision line 4 formed in the side member of the stent body. In some embodiments, the incision line may be opened by the sharp parts 46 of the nut 30, as described above. In some embodiments, the incision line is formed as two arc spanning about 90 degrees. The incision line is positioned adjacent to the gum region when the stent body is disposed over at least one patient's tooth 2. Arrows 3 may illustrate rotation of the sharp parts 46 of the nut 30, as described above, when the assembly 70 is used with the stent of FIG. 7.

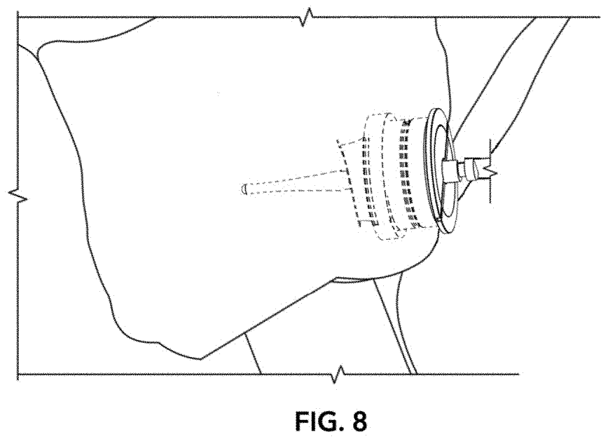

FIG. 8 illustrates the use of the assembly 70 on a patient's soft tissue, as described above.

A stent may be applied to a patient's teeth and a surgical guide may be used to provide access to the soft tissue and bone below a tooth of interest. The below describes the use of such a system, including an alignment guide for ensuring consistent and accurate placement of retro filling material.

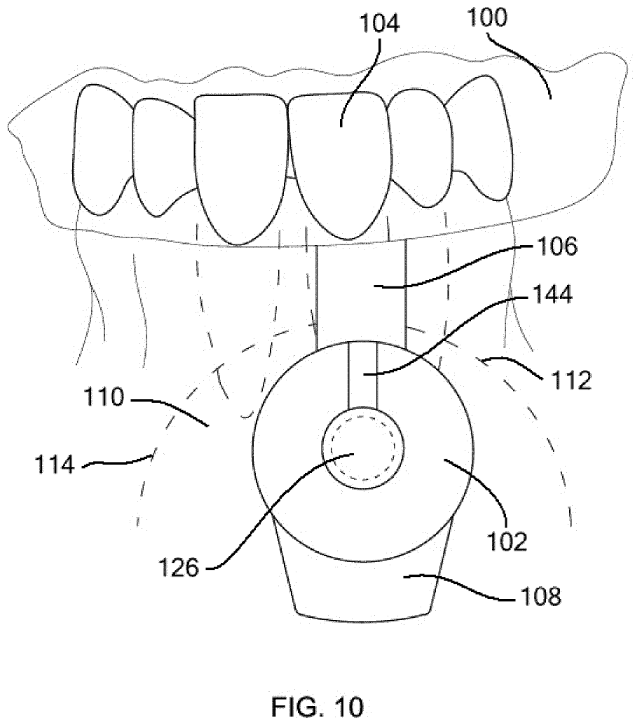

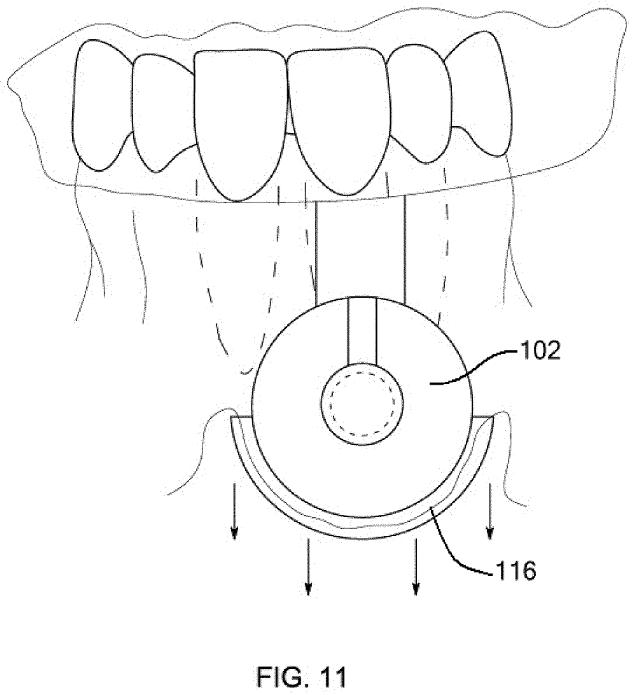

Referring now to FIGS. 10 and 11, a stent 100, including a surgical guide 102, can be test fitted on the patient's supporting teeth. A connector 106 can join the stent 100 with the surgical guide 102. The size of the system can vary, depending on which tooth is being worked on. For example, different sizes may be provided for anterior teeth, pre-molars and molars. Typically, the surgical guide 102 can fit well and is stable enough to resist any rocking motion faciolingually and mesiodistally. While there might be a little rocking faciolingually due to soft tissue, typically this is resolved after reflecting the flap, as discussed below, and adapting the stent to the bone.

For most surgical procedures, anesthetic approaches are conventional. In most regions, a block is administered. Then local infiltration of an anesthetic with 1:50,000 epinephrine is given to enhance hemostasis. A long-acting anesthetic agent is recommended, such as bupivacaine or etidocaine. Bupivacaine 0.5% with epinephrine 1:200,000 has been shown to give long-lasting anesthesia and later provide lingering analgesia.

A properly designed and carefully reflected flap 110 can result in proper access and post-op healing. The basic principles of full thickness, semi-lunar flap design should be followed. The surgical guide 102 can be placed on the patient and arced incisions, for example, a 120° clockwise incision 112 and a 120° anti-clockwise incision 114, can be made with blade #15 to the alveolar mucosa around the surgical guide 102.

The surgical guide 102 can be removed and a firm incision should be made through periosteum to alveolar bone connecting the two previous incisions 112, 114. Typically, one wants to incise and reflect a full-thickness flap to minimize hemorrhage and to prevent tearing of the tissue.

The surgical guide 102 can be placed gently on the teeth 104 so that the tissue 116 is directed towards the back of the tissue separator 108. The tissue separator 108 may be a rigid or semi-rigid member disposed at a side of the surgical guide 102 opposite the stent 100. The tissue separator 108 may be configured to be inserted into the incision and separate the external tissue so that it may be reattached over a hole drilled thereunder. If hemorrhage from soft or hard tissue is excessive to the extent that visibility is compromised, homeostatic agents or other techniques are useful.

After putting the surgical guide 102 on the patient's teeth 104, the patient should bite the surgical guide 102 and keep it for stability. Optionally, the patient can bite on a bite block placed on the teeth 104.

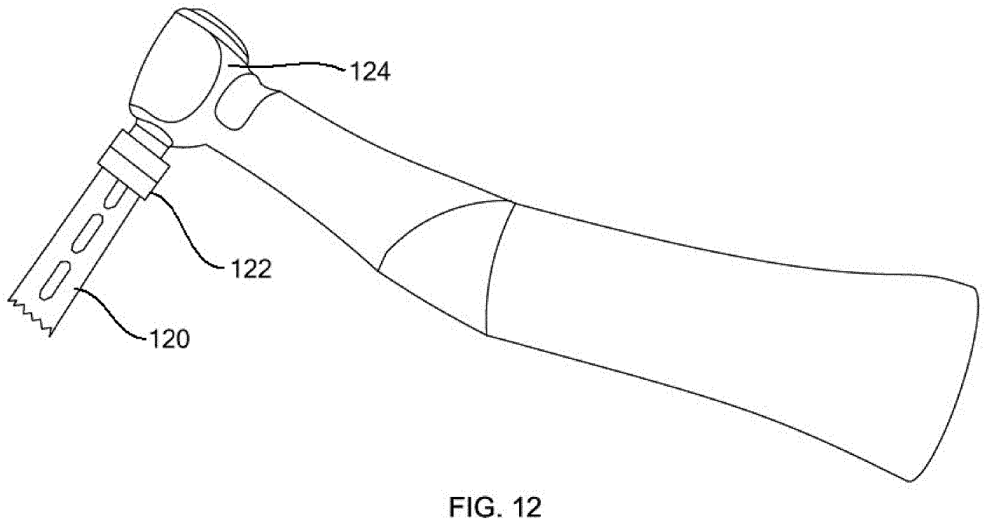

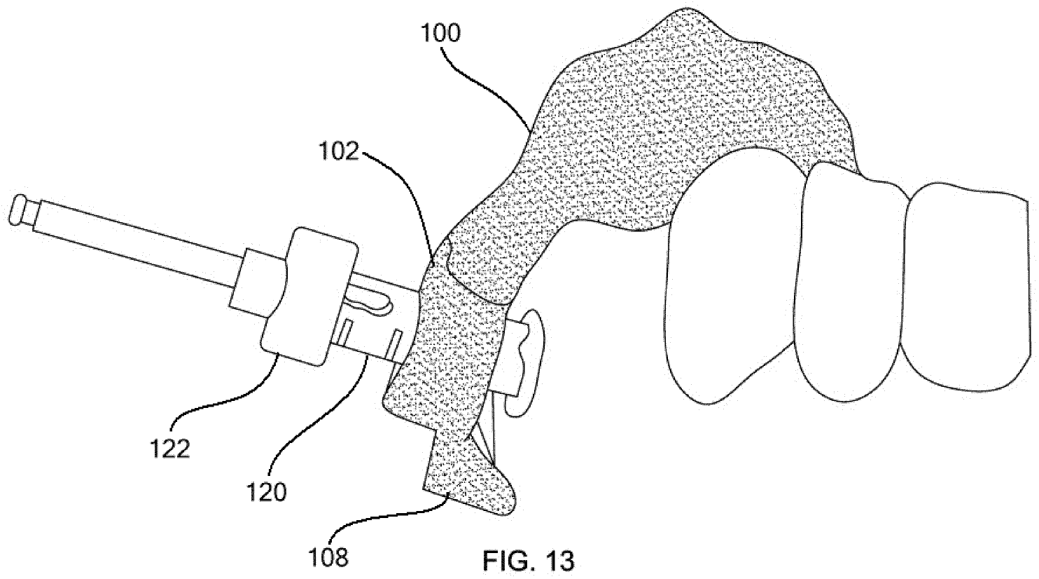

At this point, everything is ready for the surgery. Before using the trephine 120 bur, as shown in FIG. 12, a stopper 122 should be set on the trephine tip and then lock the butt in the handpiece 124. There are different sizes for the trephine bur 120. Typically, a recommended size is provided depending on which tooth is being treated.

The trephine bur 120 with the stopper 122 attached to it, can be inserted into a central hole 126 of the surgical guide 102 (see FIG. 10). The trephine bur 120 can be used to drill through bone until the stopper 122 touches the stent body (the exterior of the surgical guide 102, adjacent the hole 126). The stopper 122 can be designed according to the desired working length.

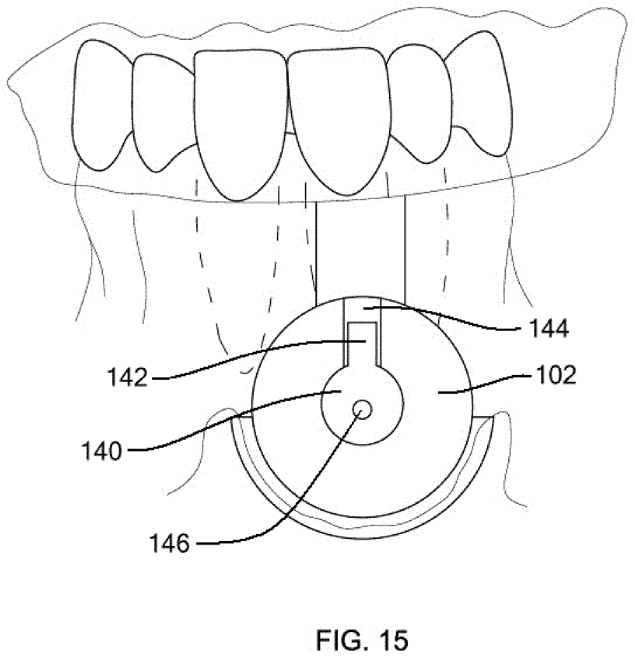

Bone and root tip, infected tissue or foreign material can be removed. Usually the hard tissue is stuck inside the trephine bur. If, in some cases, there are debris from root tip or bone left in the surgical site, it can be removed with a bone curette or bur. The surgical site can be flushed with copious amounts of sterile saline to remove soft and hard tissue debris, hemorrhage, blood clots, and excess root end filling material (if present from a prior endodontic procedure). FIG. 15 illustrates the trephine bur 120 used in a drilling operation.

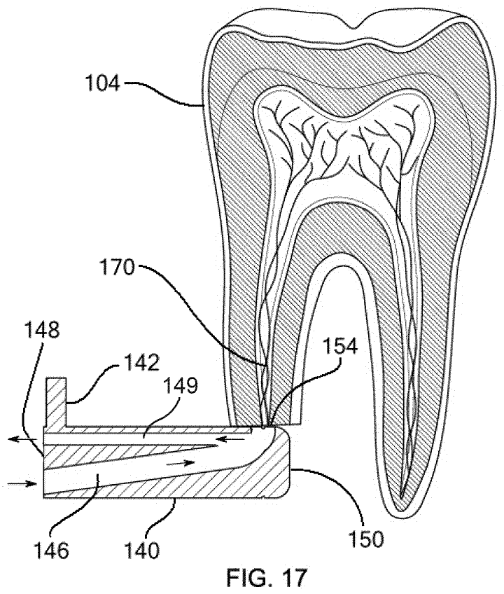

Before manipulation of the root, such as a perforation repair, crack repair or root end cavity preparation, and insertion of injecting the retro filling material in the canal, a radiograph is taken to verify that the surgical objectives are satisfactory. If corrections are needed, these are made. In cases of endodontic surgery, the cut root end surface may be prepared for a cavity into the root to include the canal or other configurations such as a dome preparation. (see FIG. 17).

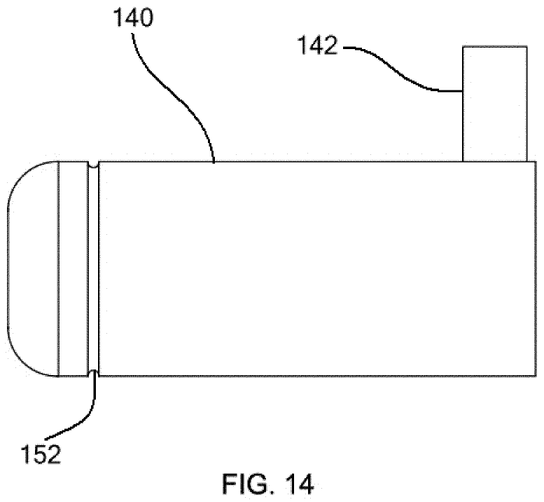

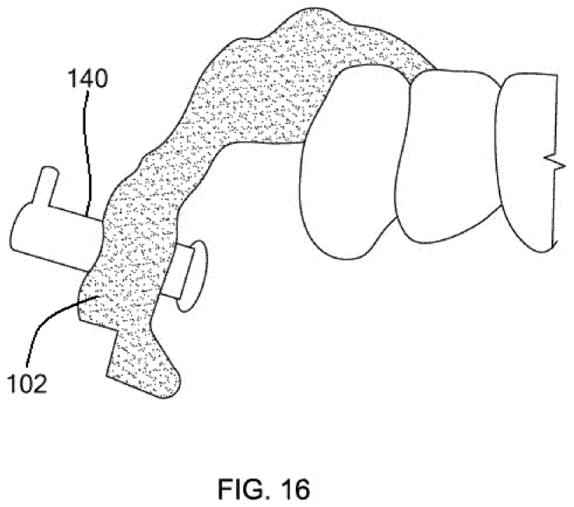

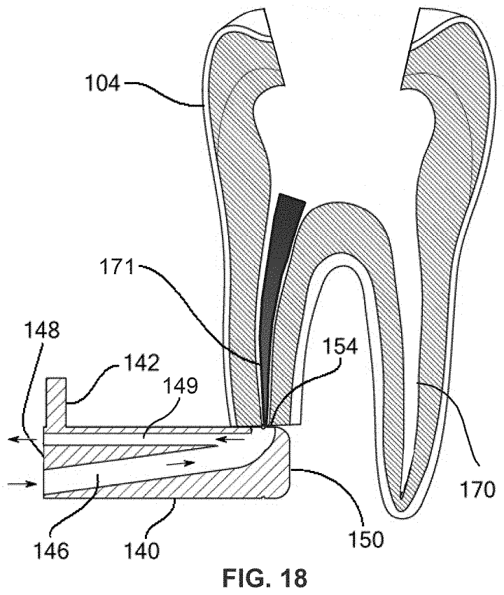

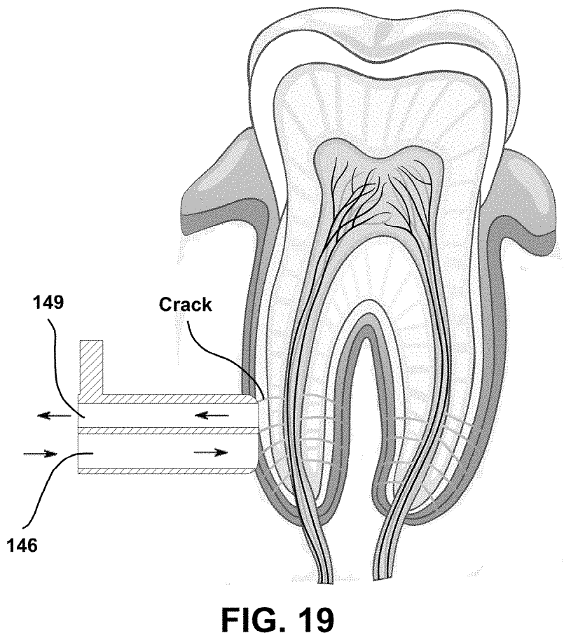

Referring now to FIGS. 14 through 19, an alignment suction sleeve 140 (also simply referred to as sleeve 140, or alignment sleeve 140) can be inserted in the surgical guide hole 126 (see FIG. 10), in the way that a male part 142 of sleeve 140 fits in the female part 144 of surgical guide 102. The sleeve 140 includes at least a first bore 146 extending therethrough from a proximal end 148 to a location at the side of the sleeve 140, near a distal end 150 of the sleeve 150. A second bore 149, typically for suction, can extend from the proximal end 148 to connect with the first bore 146 near its distal end. In some embodiments, as shown in FIG. 19, the first bore 146 and the second bore 149 may terminate at the distal end 150 of the sleeve 150, thereby permitting access to a side of a tooth root to repair, for example, a cracked root. In some embodiments, the bore 146 can include a radio-opaque part to assist in following on an x-ray pattern.

The length of the sleeve 140 may be configured so that the end of the bore 146 is positioned at any site within the trephinated cavity including on the root canal space immediately below the root of the tooth of interest, as shown in FIG. 17. The bore size can also be of different sizes to be adjacent to any part of the trephinated cavity/root surface including adjacent to a part/all or a larger area of the root end. In certain cases the root surface may be manipulated by mechanical, thermal or laser devices to change its characteristics. In certain cases, a rotary file/bur, sonic, pieso, laser or ultrasonic tip (not shown) can be inserted through the bore 146 and into the canal 170 for preparing the root surface or canal space from the arrow section of FIG. 17. In certain cases, dental materials can be placed in the canal 170, as discussed below. The sleeve 140 may include a groove 152 or radiographic marker about its circumference where the bore 146 exits at the side of the sleeve 140. This groove or radiographic marker 152 may be useful as a guide for X-Ray pattern where doctors can see the bore hole of the sleeve and help with its alignment and position within the trephined cavity. as shown in FIG. 17.

In cases where a root end filling 171 may need to be placed, according to the CBCT image, the end 154 of the bore 140 of the sleeve 140 is designed to be exactly next to the canal location. Root end preparation can be done by rotary files/bur, sonic, piezo, laser or ultrasonic tip.

Once the canal 170 is prepared, the retro-filling materials (not shown) may be placed in the canal. Before inserting the filling materials in the canal, a radiograph can be made to verify that the canal is properly prepared. If corrections are needed, these are made before inserting root end filling material. A tip set to the measured length can be used to place the root end filling materials.

The sleeve 140 and the surgical guide 102 can be removed and the surgical site can be flushed with copious amounts of sterile saline to remove soft and hard tissue debris, hemorrhage, blood clots and excess root end filling material. Bone graft material can be mixed with sterile saline and applied to the surgical site until the hole is filled.

To conclude the procedure, the flap can be returned to its original position and held with moderate digital pressure and moistened gauze. This expresses hemorrhage from under the flap and initial adaptation and more accurate suturing. Sutures are generally used, such as absorbable 4-0 suture. After suturing, the flap should again be compressed digitally with moisten gauze for several minutes to express more hemorrhage. The suture knots should note be too tight or it may strangle the tissue and decrease blood supply and cause hypertrophic scars. This encourages less postoperative swelling and more rapid healing.

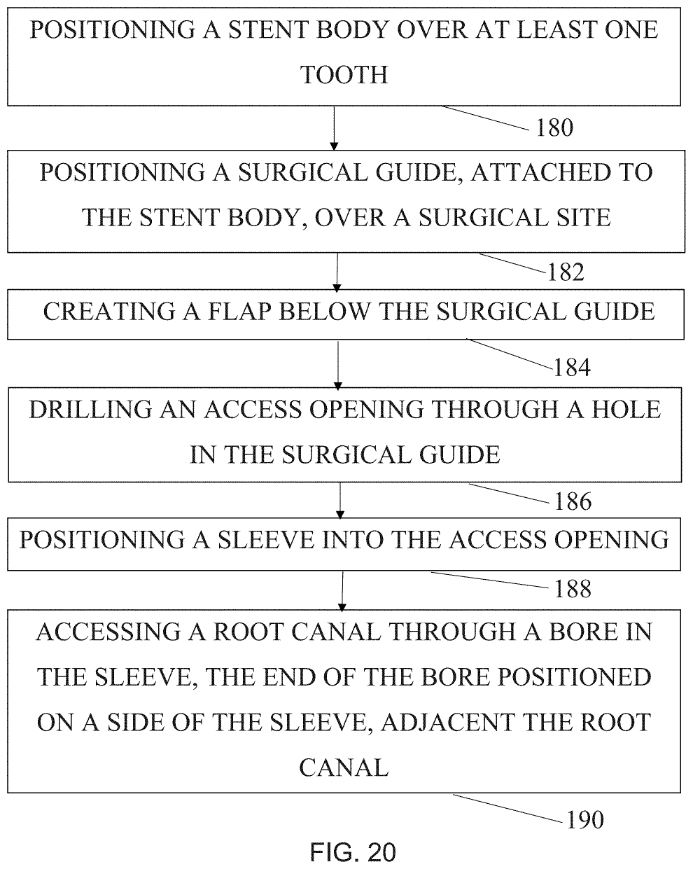

Referring to FIG. 20, the above method is summarized in a flow chart. At step 180, a stent body is positioned over at least one tooth. At step 182, the surgical guide, attached to the stent body, is positioned. At step 184, a flap is created below the surgical guide. The flap may be formed as discussed in detail above. At step 186, an access opening is formed, typically with a trephine bur, through a hole/opening in the surgical guide. At step 188, a sleeve is positioned into the access opening. At step 190, a root canal of the tooth is accessed through a bore in the sleeve. This access can be for cleaning and filling the root canal.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Claim elements and steps herein may have been numbered and/or lettered solely as an aid in readability and understanding. Any such numbering and lettering in itself is not intended to and should not be taken to indicate the ordering of elements and/or steps in the claims.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different ones of the disclosed elements.

The words used in this specification to describe the invention and its various embodiments are to be understood not only in the sense of their commonly defined meanings, but to include by special definition in this specification the generic structure, material or acts of which they represent a single species.

The definitions of the words or elements of the following claims are, therefore, defined in this specification to not only include the combination of elements which are literally set forth. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what incorporates the essential idea of the invention.

What is claimed is:

1. A dental surgical system comprising:
a stent body configured to drape over at least one tooth;
a surgical guide attached to one side of the stent body, the surgical guide positioned over at least a portion of a gum region of a tooth;
an opening disposed through the surgical guide;
a sleeve configured to fit into the opening of the surgical guide; and
one or more bores through the sleeve, the one or more bores communicating a proximal end of the sleeve with a one of a side of the sleeve, adjacent a distal end thereof, or the distal end of the sleeve.

2. The dental surgical system of claim 1, further comprising:
a channel formed on an exterior surface of the surgical guide, the channel extending from the opening toward an external periphery thereof; and
a male part extending from the proximal end of the sleeve, the male part aligning with the channel when the sleeve is inserted into the surgical guide.

3. The dental surgical system of claim 1, further comprising a groove formed about a circumference of the sleeve, the groove intersecting a position where the bore exits the sleeve at the side of the sleeve.

4. The dental surgical system of claim 1, further comprising a tissue separator positioned at side of the surgical guide opposite the stent body.

5. The dental surgical system of claim 1, further comprising a trephine bur operable to fit into the opening in the surgical guide to drill an access opening to a base of a tooth, the access opening providing access into a canal of the tooth.

6. The dental surgical system of claim 5, wherein the sleeve is configured to fit into the opening such that an end of the bore through the sleeve aligns with any part of the root.

7. The dental surgical system of claim 5, further comprising a stopper one the trephine bur, the stopper limiting a depth of the access opening, the stopper contacting the surgical guide to prevent the trephine bur from extending the depth beyond a required depth.

8. A method for accessing a root canal of a tooth comprising:
draping a stent body at least one tooth, the stent body having a surgical guide attached to one side thereof, the surgical guide positioned over at least a portion of a gum region of the tooth;
creating an incision through tissue near a desired access site;
flapping the tissue at the incision;
positioning the surgical guide under the flap;
positioning a trephine bur through an opening in the surgical guide;
forming an access opening through tissue and bone with the trephine bur;
terminating the access opening at a root of the tooth; and
inserting a sleeve into the access opening, the sleeve having a plurality of bores through the sleeve, the bores communicating a proximal end of the sleeve with either a side of the sleeve adjacent a distal end thereof or the distal end of the sleeve, the bores positioned adjacent the root of the tooth.

9. The method of claim 8, wherein the step of creating the incision includes:
creating first and second incisions on each side of the surgical guide; and
removing the surgical guide to form an incision joining the first and second incisions.

10. The method of claim 8, wherein the step of flapping the tissue includes inserting a tissue separator into the incision and moving the tissue separator away from the tooth to create the flap.

11. The method of claim 10, wherein the tissue separator is attached to the surgical guide opposite the stent body.

12. The method of claim 8, wherein the step of inserting the sleeve into the access opening includes aligning a male part, extending from the proximal end of the sleeve, with a channel formed on an exterior surface of the surgical guide, the channel extending from the opening toward an external periphery thereof.

13. The method of claim 8, further comprising inserting dental tool through the bore to prepare the root surface or the root canal.

14. The method of claim 8, further comprising inserting a filling material through the bore to be applied into the root canal or manipulating a root mechanical, thermal or laser device.

15. The method of claim 8, further comprising visualizing a proper depth of the sleeve in the access opening with an x-ray view showing a groove, formed about a circumference of the sleeve and intersecting a position where the bore exits the sleeve at the side of the sleeve, aligning with the root canal when the proper depth is realized.

16. The method of claim 15, wherein the sleeve includes a radiographic mark as a cutout in an outer periphery of the sleeve at the position where the bore exits the sleeve.

17. A method for treating a tooth, comprising:
draping a stent body at least one tooth, the stent body having a surgical guide attached to one side thereof, the surgical guide positioned over at least a portion of a gum region of the tooth;
creating a first incision and a second incision at each side of the surgical guide; removing the surgical guide to provide a further incision connecting the first incision with the second incision;
flapping the tissue at the incision with a tissue separator, attached to the surgical guide opposite the stent body, as the surgical guide is positioned under the flap;
positioning a trephine bur through an opening in the surgical guide;
forming an access opening through tissue and bone with the trephine bur;
terminating the access opening at a root of the tooth;
inserting a sleeve into the access opening, the sleeve having a plurality of bores through the sleeve, the bores communicating a proximal end of the sleeve with a side of the sleeve adjacent a distal end thereof, the bore positioned adjacent the root of the tooth;
inserting a dental tool through the bore to clean the root canal; and
inserting a retro filling material through the bore to be applied into the root canal.

18. The method of claim 17, wherein the step of inserting the sleeve into the access opening includes aligning a male part, extending from the proximal end of the sleeve, with a channel formed on an exterior surface of the surgical guide, the channel extending from the opening toward an external periphery thereof.

19. The method of claim 17, further comprising visualizing a proper depth of the sleeve in the access opening with an x-ray view showing a groove, formed about a circumference of the sleeve and intersecting a position where the bore exits the sleeve at the side of the sleeve, aligning with the root canal when the proper depth is realized.
